(19) United States
(12) Patent Application Publication
(10) Pub. No.: US 2012/0271261 A1
(43) Pub. Date: Oct. 25, 2012

(54) Breast and Body Crease Sweat and Moisture Absorb

(76) Inventor: Larry Lofton, Rancho Cucamonga, CA (US)

(21) Appl. No.: 13/092,780

(22) Filed: Apr. 22, 2011

(51) Int. Cl. A61F 13/45; A61L 15/22

(52) U.S. Cl. 604/367; 604/385.01

(57) ABSTRACT

A moisture absorbing pad adapted for insertion into creases of the body prone to perspiration is made of a generally planar sheet of moisture absorbing material. The planar sheet is formed into two parts, a first part rolled into a cylinder having two ends which are substantially flattened, and a second part folded back on itself to form a tab, which is substantially teardrop shaped in profile. The material may have anti-microbial or emollient properties. To use the pad, a body crease is opened and the pad inserted near the center of the crease. The crease may then be reformed around the pad. When inundated with moisture, the pad may be extracted by using the tab and flattened ends as handles.

### BACKGROUND

In warm climates and heated environments, creased areas of the body wherein one portion of a person's skin overlays and is in contact against another, frequently causes excessive perspiration and discomfort. In extreme cases, when a perspiration prone area remains unexposed or unventilated for a prolonged period, adverse health effects occur. There is consequently a need in the art for a body crease moisture absorbing device, designed for easy application and removal, which is inexpensive to manufacture.

### SUMMARY

A moisture absorbing pad is formed of a sheet of moisture absorbing material. The sheet is formed into two parts, a first cylindrical roll of material, and a second area of the sheet folded back upon itself to form a tab, teardrop shaped in profile. The cylindrical roll has substantially flattened ends. The material may be constructed of cloth-like paper to impart tear resistance properties, may have anti-microbial properties, either by the nature of the material itself, or having been treated with an anti-microbial agent, and the material may also include an emollient for treating irritated areas.

The flattened ends and tab are oblong to form handles from which the moisture absorbing pad can be grasped and removed. The flattened ends are parallel to each other, forming complimentary handles. To use the moisture absorbing pad, a creased area of skin is unfolded, and the pad placed against the skin parallel to and near the center of the crease. The skin can then be released, reforming the crease. The moisture absorbing pad may be worn until inundated with moisture, at which time a user grasps the tab, one of the flattened ends, or both, and removes the pad from the treated area.

### BRIEF DESCRIPTION OF THE FIGURES

FIG. 1 is a front view of a moisture absorbing pad.

FIG. 2 is a perspective view of a moisture absorbing pad showing substantially flattened ends and a looped portion.

FIG. 3 is a cut-away perspective view of a moisture absorbing pad showing the spiral configuration of the pad.

FIGS. 4 and 5 are left and right side views, respectively, of a moisture absorbing pad.

### DESCRIPTION

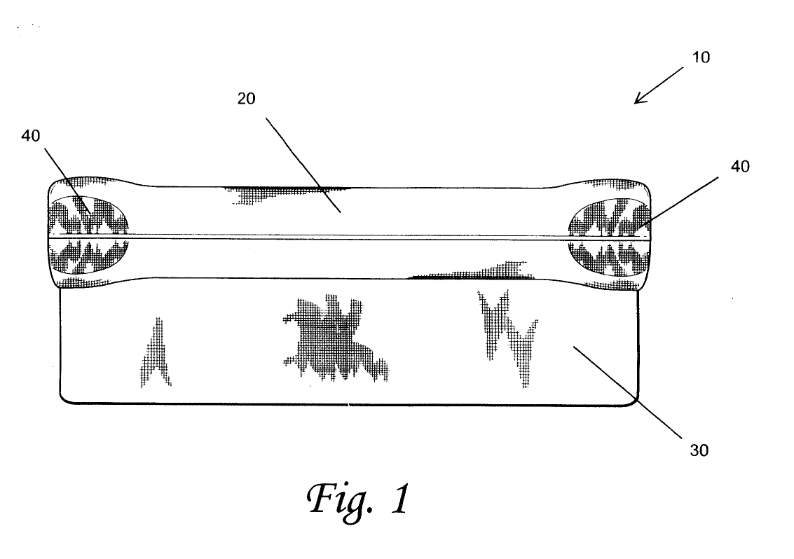

Referring to FIG. 1, a moisture absorbing pad is shown and described. The pad 10 is comprised of a substantially planar member of moisture absorbing material, separated into two regions. A first region 20, comprising a portion of the material rolled into a cylinder is connected to a second region 30, comprising a portion of the material folded back upon itself to form a tab of material teardrop shaped in profile. The first region 20 comprises two substantially flattened ends 40. In one embodiment, the flattened ends 40 are flattened parallel to each other.

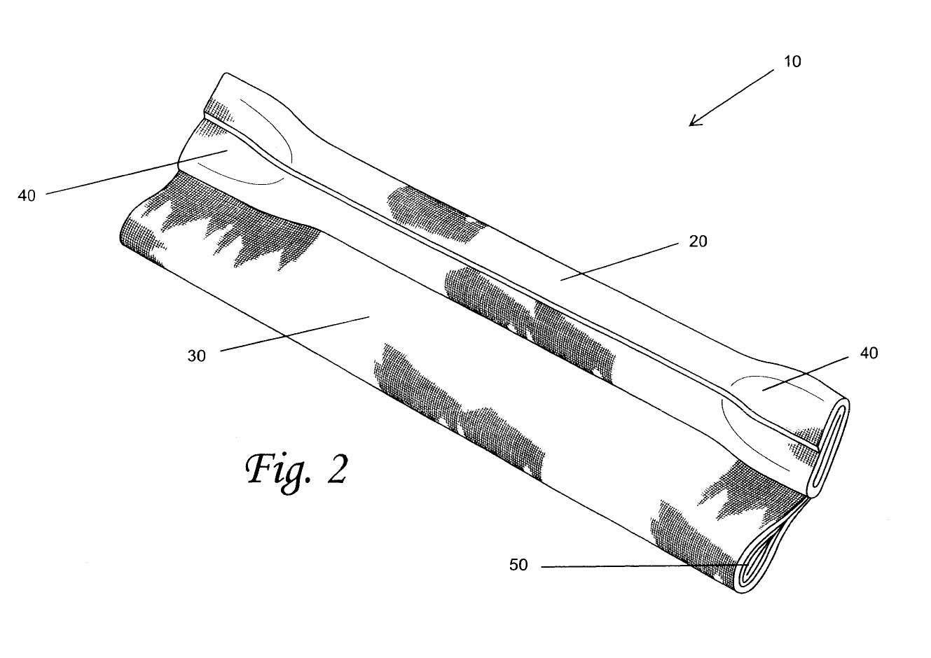

Referring to FIG. 2, a perspective view of the moisture absorbing pad 10 is shown. In this view, side details of the flattened ends 40 of the first region 20 and second region 30 are shown. The second region end 50 is loosely folded back upon itself to create a loop without forming a fold or crease.

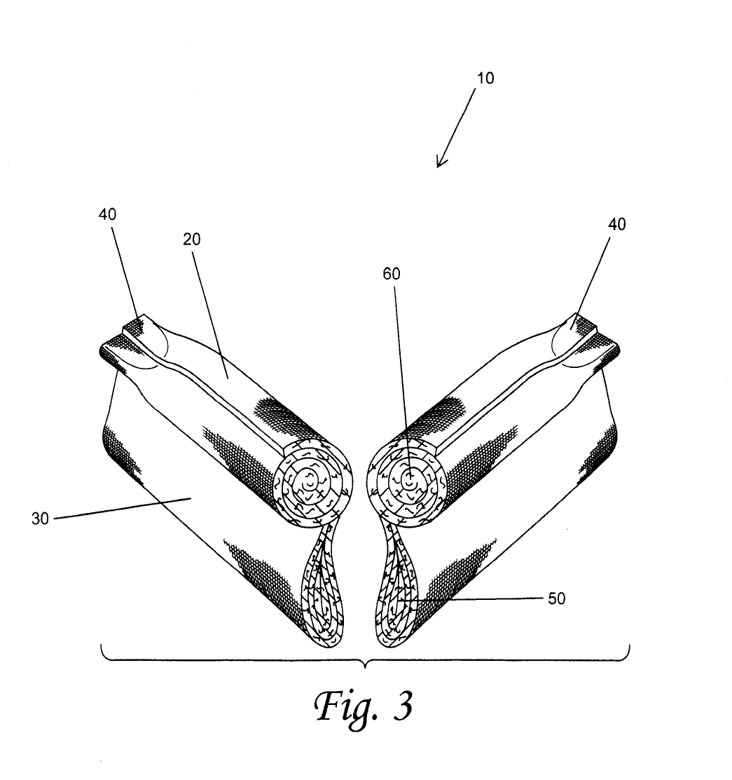

Referring to FIG. 3, a cut-away perspective view of the moisture absorbing pad 10 is shown. In this view, the rolled nature of the first region 20 is depicted wherein the material is rolled into a spiral configuration 60. The flattened ends 40 and teardrop shape 50 of the second region 30 are also shown. The moisture absorbing pad 10 may be constructed of or comprise a cloth-like paper material to impart tear resistance properties to the material. The material of the moisture absorbing pad 10 may also comprise anti-microbial properties, either by the nature and construction of the material itself, or the material having been treated with an anti-microbial compound. In another embodiment, the material may comprise an emollient for treating irritated areas.

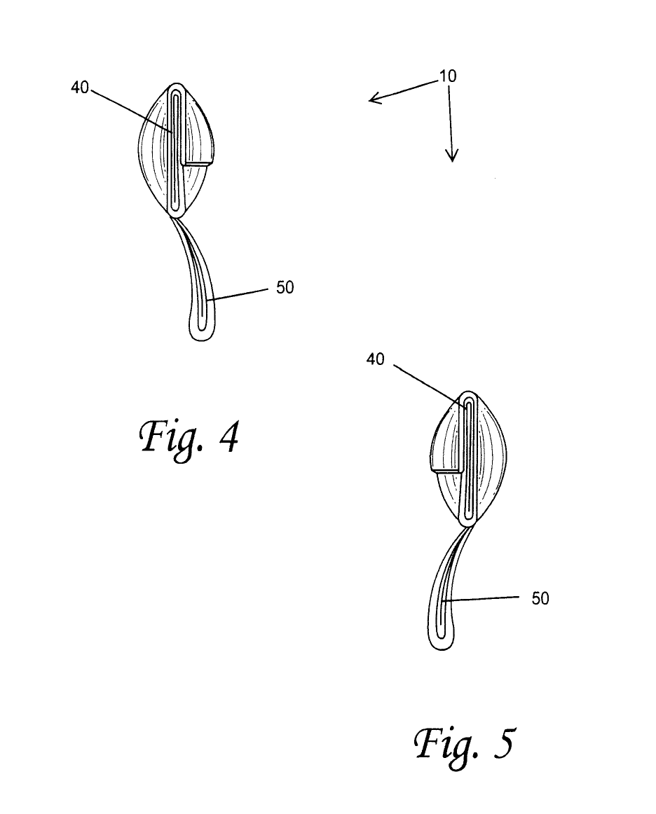

Referring to FIGS. 4 and 5, side views of the moisture absorbing pad 10 are shown. In these views, the oblong nature of the flattened ends 50 and second region 30 are depicted. It is anticipated that the moisture absorbing pad 10 will need to be removed and replaced periodically. In furtherance of this objective, the second region 30 and flattened ends 50 form substantially handle-shaped areas, from which the moisture absorbing pad 10 may be grasped for removal. In one embodiment, the flattened ends 50 are formed parallel to each other to form complimentary handles.

To employ the moisture absorbing pad 10, a user manipulates a substantially creased area of skin into an open and generally flat configuration. The first region 20 of the moisture absorbing pad 10 is placed against the skin, parallel to the crease, approximately near the center fold of the crease. The skin is then released, causing the crease to reform over the first region 20. The moisture absorbing pad 10 may be worn until inundated with moisture, at which time a user grasps either the second region 30 or one of the flattened ends 40, and removes the moisture absorbing pad 10 from the treated area. It is anticipated that a user may need to once again manipulate the creased area into an open, generally flat configuration to accomplish removal.

While a particular form of the invention has been illustrated and described, it will be apparent that various modifications can be made without departing from the spirit and scope of the invention. For example, structure, materials, procedure and the like that will fall within the scope of the invention. Accordingly, it is not intended that the invention be limited, except as by the appended claims.

What is claimed is:

1. A moisture absorbing pad comprising:
a first elongate portion of moisture absorbing material formed in a generally rolled cylinder shape having substantially flattened opposing ends; and
a second elongate portion of moisture absorbing material folded back upon itself to form a looped tab, generally teardrop shaped in profile, the second portion attached along the length of the first portion, whereby the second portion and the opposing ends may be used as handles for insertion and removal of the pad.

2. The moisture absorbing pad of claim 1 wherein the material of the first elongate portion comprises a cloth-like paper material.

3. The moisture absorbing pad of claim 1 wherein the material of the first elongate portion comprises anti-microbial properties.

4. The moisture absorbing pad of claim 1 wherein the material of the first elongate portion comprises an emollient.

5. A method of employing a moisture absorbing pad having a generally cylindrical portion and a handle portion comprising the steps of:
manipulating a creased area of a user's skin to form a generally flat area;
placing the cylindrical portion of the moisture absorbing pad parallel to and proximal the center of the creased area;
releasing the skin to re-form the crease, wherein the cylindrical portion of the moisture absorbing pad is held in place by the skin; and
removing the moisture absorbing pad when desired by pulling the handle portion away from the crease.
